(19) United States
(12) Patent Application Publication
(10) Pub. No.: US 2005/0228038 A1
(43) Pub. Date: Oct. 13, 2005

(54) 11-Beta hydroxysteroid dehydrogenase type 1 inhibitors as anti-obesity/anti-diabetes compounds and 17-beta hydrosteroid dehydrogenase type I inhibitors as useful agents for the treatment of cancers, especially breast cancer

(76) Inventors: David L. Vander Jagt, Albuquerque, NM (US); Robert E. Royer, Albuquerque, NM (US); Lorraine M. Deck, Albuquerque, NM (US)

Correspondence Address: Henry D. Coleman, 714 Colorado Avenue, Bridgeport, CT, 06605, US

(21) Appl. No.: 11/093,493

(22) Filed: Mar. 30, 2005

Related U.S. Application Data
(60) Provisional application No. 60/560,387, filed on Apr. 8, 2004.

(51) Int. Cl. A61K 31/353; A61K 31/192; A61K 31/075

(52) U.S. Cl. 514/451; 514/569; 514/715

(57) ABSTRACT

This invention is directed to the discovery that 11-Beta Hydroxysteroid Dehydrogenase Type 1 may be a common molecular etiology for visceral obesity and the metabolic syndrome of obesity as well a treatment for diabetes, especially type II diabetes. The present invention also relates to the use of certain compounds as inhibitors of 17-Beta Hydroxysteroid Dehydrogenase Type 1 and their use for the treatment of cancer, especially breast cancer.

### RELATED APPLICATIONS

This application claims priority from U.S. provisional application US 60/560,387, filed Apr. 8, 2004, the entire contents of which is incorporated by reference herein.

This invention is directed to the discovery that 11-Beta Hydroxysteroid Dehydrogenase Type 1 is a common molecular etiology for visceral obesity and the metabolic syndrome of obesity as well a treatment for diabetes, especially type II diabetes. The present invention also relates to the use of certain compounds as inhibitors of 17-Beta Hydroxysteroid Dehydrogenase Type 1 and their use for the treatment of cancer, especially breast cancer, including drug resistant breast cancer.

### BACKGROUND OF THE INVENTION

Obesity is a devastating disease. In addition to harming physical health, obesity can wreak havoc on mental health because obesity affects self-esteem, which ultimately can affect a person's ability to interact socially with others. Unfortunately, obesity is not well understood, and societal stereotypes and presumptions regarding obesity only tend to exacerbate the psychological effects of the disease. Because of the impact of obesity on individuals and society, much effort has been expended to find ways to treat obesity, but little success has been achieved in the long-term treatment and/or prevention of obesity.

The adverse metabolic consequences of obesity are best predicted by the quantity of visceral fat. Excess glucocorticoids produce visceral obesity and diabetes, but circulating glucocorticoid levels are normal in typical obesity. Glucocorticoids can be produced locally from inactive 11-keto forms through the enzyme 11-Beta-hydroxysteroid dehydrogenase type 1 (11 HSD-1).

Breast cancer is the most prevalent cancer in the United States, with 203,500 projected new cases in 2002, accounting for ˜31% of all new cancers in women, according to the American Cancer Society. It also accounts for ˜30% of all new cancers in women in Canada, according to the National Institute of Canada, with 19,500 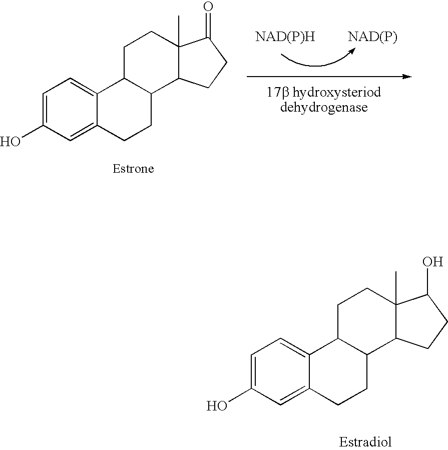
estimated new cases in 2001, where it is the number one cancer in women. In malignant breast tissue the concentration of 17-beta-estradiol is significantly increased and 17βestradiol is known to stimulate the growth of breast cancer cells. 17-beta-Hydroxysteroid dehydrogenase type I (17betaHSD1) catalyzes the NADPH dependent reduction of estrone into estradiol, which is biologically active. Therefore, limiting estradiol production by inhibition of 17betaHSD is an approach to the design of therapeutics for breast cancer.

Obesity is an epidemic and chronic disease that affects nearly one-third of the adult American population (approximately 60 million) causing 300,000 deaths in the United States. It can be linked to 30 medical conditions including high blood pressure, diabetes, heart disease, stroke and cancer of the breast. In adipose tissue the activity of 11betahydroxysteroid Dehydrogenase type I (11betaHSD1) is high resulting in increased 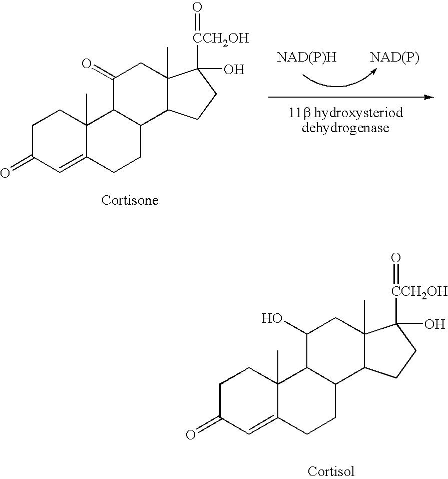
levels of cortisol and elevated levels of visceral adipose tissue. 11betaHSD1 catalyzes the NADPH dependent conversion of cortisone to cortisol. Inhibition of 11betaHSD1 within the adipose tissue will offer potent therapy for central obesity.

In 1975 Rossmann characterized the dinucleotide binding site of several dehydrogenases among which is lactate dehydrogenase (LDH). He defined the classical Rossmann fold for two domain proteins resembling LDH. The minimum structure needed to bind the cofactor is a βαβαβ unit with an additional β strand associating with this unit to form the “core” topology. There is a fingerprint region in this core topology which is the first 30-35 amino acids. The characteristics of this fingerprint region are a phosphate binding consensus sequence, a conserved negatively charged residue at the end of the second β strand, a conserved positively charged residue at the beginning of the first β strand, and six positions occupied by small hydrophobic amino acids. The phosphate binding sequence is a glycine-rich consensus sequence GXGXXG. There is a hydrogen bond between the cofactor and the protein, specifically the pyrophosphate oxygen of NADP and the carboxy-terminal glycine of the first a helix of the babab unit.

The short chain dehydrogenase/reductase enzymes, which include 11betaHSD1 and 17betaHSD1, represent a family of dehydrogenases with a modified Rossmann fold. The modified Rossmann fold contains seven or eight b strands rather than the normal six. It is located near the N-terminus and the signature motif is different, GXXXGXG, but has the same function for cofactor binding. Shown below is a crystalline complex of mutant 17betaHSD1, estradiol, NADP containing two monomers and two Rossmann-fold motifs depicted in light pink and light blue. The motifs are labeled bA-aB-bB-aC-bC-aD-bD. Since 11betaHSD1 has not been crystallized it is assumed to have a modified Rossmann fold because of the high homology to several other crystallized HSD proteins.

We have demonstrated previously in our laboratory that all isozymes of lactate dehydrogenase, having a classical Rossmann fold, can be selectively inhibited by derivatives and analogs of the natural product gossypol. Inhibition is competitive with the binding of NAD(P)H. We have also demonstrated through computational design that appropriate incorporation of a substrate analog into the Rossmann fold inhibitors may provide increased potency. Thus, analogs and derivatives of gossypol may serve as lead compounds for design of inhibitors of dehydrogenases that possess a Rossmann fold.

### SUMMARY OF THE INVENTION

Known inhibitors of 17betaHSD1 and 11betaHSD1 are generally steroid structures and possess undesired biological activitites. Both enzymes are members of the short-chain dehydrogenase/reductase family (SDR) and they bind NADPH in a motif that is described as a modified Rossmann fold. We have shown that enzymes having classical Rossmann folds can be selectively inhibited by derivatives and analogs of the natural product gossypol. Therefore we suggest that derivatives of gossypol may represent attractive lead compounds for the design of potent and selective inhibitors of 17betaHSD1 and 11betaHSD1 resulting in therapeutics to treat breast cancer and obesity and related conditions and/or disease states.

Consequently, the present invention provides methods of treating obesity or diabetes, especially type II diabetes, the methods comprising the step of administering to an obese or diabetic patient or a patient at risk of becoming obese or become diabetic a therapeutically effective amount of a compound that is a 11 HSD-1 inhibitor.

Also provided are methods of treating obesity, the methods comprising the step of administering to an obese patient or a patient at risk of becoming obese a therapeutically effective amount of a compound that is a selective 11 HSD-1 inhibitor.

Also provided are pharmaceutical compositions comprising:
a) a compound that is a 11 HSD-1 inhibitor; and
b) an effective amount of a second compound useful for the treatment of obesity, diabetes (especially type II diabetes), atherosclerosis, insulin resistance, impaired glucose tolerance, hypercholesterolemia or hypertrigylceridemia. Such compounds include statin compounds, for example, lovastatin (Mevacor), pravastatin sodium (Pravachol), simvastatin (Zocor), fluvastatin sodium (Lescol, Lescol XL), atorvastatin calcium (Lipitor), rosuvastatin (Crestor), ezetimibe/simvastatin (Vytorin), among others, including clofibrate, gemfibrozil and niacin, among others.

The present invention provides methods of treating obesity, the methods comprising the step of administering to an obese patient or a patient at risk of becoming obese a therapeutically effective amount of a compound that is an 11-Beta-hydroxysteroid dehydrogenase type 1 inhibitor.

The present invention also relates to compounds and pharmaceutical compositions which function as inhibitors of 17BHSD1 and as useful therapeutic agents in the treatment of cancer, especially breast cancer. The present method is also particularly suited for treating drug-resistant cancer, especially including drug-resistant breast cancer. Pharmaceutical compositions comprising an effective amount of one or more of the compounds of the present invention in combination with an additional anti-cancer agent for the treatment of breast cancer which has metastacized in the patient is another object of the present invention.

Also provided are pharmaceutical compositions comprising:
a) a compound that is a 17 HSD1 inhibitor; and
b) an effective amount of a second compound useful for the treatment of cancer, preferably breast cancer.

The present invention also relates to pharmaceutical compositions compising an effective amount of a compound according to the present invention in combination with another anti-cancer agent, more specifically, antimetabolites, etoposide, doxorubicin, taxol, vincristine, cytoxan (cyclophosphamide) or mitomycin C, among numerous others, including topoisomerase I and topoisomerase II inhibitors, such as adriamycin, topotecan, campothecin and irinotecan, other agent such as gemcitabine and agents based upon campothecin and cis-platin. In certain aspects of the invention, preferred additional anti-cancer agents for use in treating breast cancer in combination with one or more of the 17HSD1 inhibitors according to the present invention include doxorubicin, cyclophosphamide, methotrexate and fluorouracil, among others.

### DETAILED DESCRIPTION OF THE INVENTION

In one aspect, the present invention relates to methods of treating obesity, diabetes, atherosclerosis, insulin resistance, impaired glucose tolerance, hypercholesterolemia or hypertrigylceridemia using a compound that is an inhibitor of 11-HSD-1.

In accordance with the present invention, obesity, diabetes, atherosclerosis, insulin resistance, impaired glucose tolerance, hypercholesterolemia or hypertrigylceridemia can be treated by administering to an obese patient or a patent at risk of becoming obese or a patent having or at risk of having, diabetes, sexual dysfunction (including erectile dysfunction), atherosclerosis, insulin resistance, impaired glucose tolerance, hypercholesterolemia, or hypertrigylceridemia a therapeutically effective amount of a 11-HSD-1 inhibitor. In a preferred embodiment of the invention, the 11-HSD-1 inhibitor is a compound according to the chemical structure as described in detail hereinbelow.

The present invention is directed to compounds according to the chemical structure: 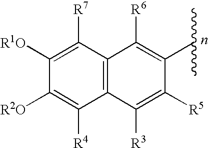
Where n is 1 or 2 such that when n is 1, n is represented as R⁹ wherein R⁹ is H, halogen, OH, OR, CO₂R group, OR₁, an optionally substituted C₁-C₈ alkyl group, an optionally substituted C₂-C₈ alkenyl or alkynyl group, an optionally substituted (CH₂)ₓcycloalkyl, (CH₂)ₓcycloalkenyl or (CH₂)ₓheterocyclic group, an optionally substituted (CH₂)ₓaryl or (CH₂)ₓheteroaryl group and when n is 2, the compound is a dimer;
R¹ and R² are independently H, an optionally substituted C₁-C₈ alkyl group, an optionally substituted C₂-C₈ alkenyl or alkynyl group, an optionally substituted (CH₂)ₓ-aryl group or (CH₂)ₓ-heteroarylgroup or a C₂-C₁₂ acyl group;
R³, R⁴ and R⁵ are independently H, halogen (F, Cl, Br, I), (CH₂)ₓOH, (CH₂)ₓCOR, (CH₂)ₓCO₂R group, (CH₂)ₓOR₁, an optionally substituted C₁-C₈ alkyl group, an optionally substituted C₂-C₈ alkenyl or alkynyl group, an optionally substituted (CH₂)ₓcycloalkyl, (CH₂)ₓcycloalkenyl or (CH₂)ₓheterocyclic group, an optionally substituted (CH₂)ₓaryl or (CH₂)ₓheteroaryl group;
R is H or an optionally substituted C₁-C₈ alkyl group;
R₁ is an optionally substituted C₁-C₈ alkyl group or C₂-C₈ acyl group or a C(O)H group;
R⁶ is H, halogen, —CN, (CH₂)ₓOH, (CH₂)ₓCOR, (CH₂)ₓCO₂R group, (CH₂)ₓOR₁, an optionally substituted C₁-C₈ alkyl group, an optionally substituted C₂-C₈ alkenyl or alkynyl group, an optionally substituted (CH₂)ₓcycloalkyl, (CH₂)ₓcycloalkenyl or (CH₂)ₓheterocyclic group, an optionally substituted (CH₂)ₓaryl or (CH₂)ₓheteroaryl group, or a 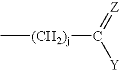
   group;
Z is N—H or O;
Y is H, OH, C₁-C₈ alkyl or O(C₁-C₈)alkyl;
R⁷ is H, halogen, —CN, (CH₂)ₓOH, (CH₂)ₓCOR, (CH₂)ₓCO₂R group, (CH₂)ₓOR₁, an optionally substituted C₁-C₈ alkyl group, an optionally substituted C₂-C₈ alkenyl or alkynyl group, an optionally substituted (CH₂)ₓcycloalkyl, (CH₂)ₓcycloalkenyl or (CH₂)ₓheterocyclic group, an optionally substituted (CH₂)ₓaryl or (CH₂)ₓheteroaryl group, or a 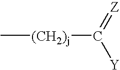
   group, or R⁷ together with R⁶ forms a 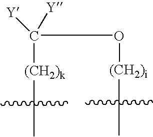
   group,
where Y′ and Y″ are each H or together are O or N—H;
each x is independently 0, 1, 2, 3, 4, 5 or 6, preferably 0 or 1;
i is 0, 1 or 2; preferably 0 or 1, more preferably 0;
j is 0, 1, 2 or 3, preferably 0 or 1, more preferably 0;
k is 0, 1, 2 or 3, preferably 0 or 1, more preferably 0; and
pharmaceutically acceptable salts thereof.

Preferred compounds which find use as inhibitors of 11 HSD-1 and as therapeutic agents for the treatment and/or prevention of obesity include, for example, compounds which are disclosed in U.S. Pat. No. 4,806,568 (Gossypol Derivatives); U.S. Pat. No. 5,026,726 (Gossylic Iminolactones and Gossylic Lactones); U.S. Pat. No. 5,780,675 (Deoxygossylic Compounds); U.S. Pat. No. 5,936,12-(Deoxygossilic compounds); and U.S. Pat. No. 6,124,498 (Hydroxynapthoic acids and derivatives), all of which patents are incorporated by reference herein. Each of the compounds disclosed therein, alone or in combination may be used to inhibit 11-HSD-1 and treat obesity accordingly.

In preferred compounds according to the present invention, R¹ and R² are each independently H, or an optionally substituted C₁-C₄ alkyl group;
R³, R⁴ and R⁵ are each independently selected from H, a halogen, an optionally substituted C₁-C₄ alkyl group, an optionally substituted —(CH₂)ₓ—O(C₁-C₄)alkyl group, an optionally substituted 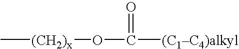
   group or 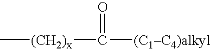
   group;
R⁹ is H, a halogen, an optionally substituted C₁-C₄ alkyl group,
an optionally substituted —(CH₂)ₓ—O(C₁-C₄)alkyl group, an optionally substituted 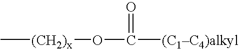
   group, a 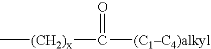
   group; an optionally substituted aryl or C₁-C₆ alkylene aryl group;
R⁶ is H, OH, a halogen, an optionally substituted C₁-C₄ alkyl group, an optionally substituted —(CH₂)ₓ—O(C₁-C₄)alkyl group or together with R⁷ forms a five or six-membered lactone or lactam ring;
R⁷ is H, a halogen, an optionally substituted C₁-C₄ alkyl group,
an optionally substituted —(CH₂)ₓ—O(C₁-C₄)alkyl group, an optionally substituted 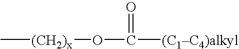
   group, an optionally substituted 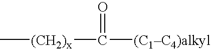
   group;
an optionally substituted aryl or C₁-C₆ alkylene aryl group or together with R6 form a five or six-membered lactone or lactam ring;
and pharmaceutically acceptable salts thereof.

The above compounds can be used to inhibit or modulate 11-β-hydroxysteroid dehydrogenase type I enzymes (11βHSD1) and 17-β-hydroxysteroid dehydrogenase type I enzymes (17βHSD1). These compounds can be used to treat obesity, diabetes, especially type II diabetes, atherosclerosis, insulin resistance, impaired glucose tolerance, hypercholesterolemia or hypertrigylceridemia and certain cancers, especially breast cancer, including drug-resistant forms of breast cancer.

The term “patient” refers to a mammal, preferably a human, in need of treatment or therapy to which compounds according to the present invention are administered in order to treat a condition or disease state modulated through the binding of a compound according to the present invention with an enzyme, and in particular, 11-β-hydroxysteroid dehydrogenase type I enzymes (11βHSD1) and 17-=62 -hydroxysteroid dehydrogenase type I enzymes (17βHSD1).

The term “modulate” means, with respect to disease states or conditions modulated through binding of a compound according to the present invention to 11-β-hydroxysteroid dehydrogenase type I enzymes (11βHSD1) and 17-β-hydroxysteroid dehydrogenase type I enzymes (17βHSD1) to produce, either directly or indirectly, an improvement or lessening of a condition or disease state which was, prior to administration of a compound according to the present invention, sub-optimal and in many cases, debilitating and even life threatening.

The term “compound” is used herein to refer to any specific chemical compound disclosed herein. Within its use in context, the term generally refers to a single compound, but in certain instances may also refer to stereoisomers and other positional isomers and/or optical isomers (including racemic mixtures) of disclosed compounds. The compounds of this invention include all stereoisomers where relevant (e.g., cis and trans isomers) and all optical isomers of the present compounds (eg., R and S enantiomers), as well as racemic, diastereomeric and other mixtures of such isomers, as well as all pharmaceutically acceptable salt forms of the present compounds, where applicable.

The term “dimer” is used herein to describe any compound where n is 2, such that the compound comprises two chemical units, each chemical unit being covalently linked to the other chemical unit.

The term “alkyl” is used herein to refer to a fully saturated monovalent radical containing carbon and hydrogen, and which may be a straight chain, branched or cyclic. Examples of alkyl groups are methyl, ethyl, n-butyl, n-heptyl, isopropyl, 2-methylpropyl, cyclopropyl, cyclopropylmethyl, cyclobutyl, cyclopentyl, cyclopentylethyl and cyclohexyl, among others. “Alkenyl” refers to an unsaturated monovalent radical group containing carbon and hydrogen. “Alkynyl” refers to a monovalent radical group containing carbon and hydrogen and at least one triple bond. “Alkylene” refers to a fully saturated hydrocarbon which is divalent (may be linear, branched or cyclic) and which is optionally substituted.

“Aryl” refers to a substituted or unsubstituted monovalent aromatic radical having a single ring (e.g., benzene) or multiple condensed rings (e.g., naphthyl, anthracenyl, phenanthryl) and can be can be bounds to compound according to the present invention at any position on the ring(s). Other examples of aryl groups include heterocyclic aromatic ring systems “heteroaryl” groups having one or more nitrogen, oxygen, or sulfur atoms in the ring, such as imidazole, furyl, pyrrole, pyridyl, indole and fused ring systems, among others, which may be substituted or unsubstituted.

“Alkoxy” as used herein refers to an alkyl group bound through an ether linkage; that is, an “alkoxy” group may be represented as —O-alkyl where alkyl is as defined above. A “lower alkoxy” group refers to an alkoxy group containing one to six, more preferably one to four, carbon atoms.

The term “cyclic” shall refer to a carbocyclic or heterocyclic group, having one to three rings, and is preferably a 5- or 6-membered ring. A heterocyclic ring shall contain at least one atom other than carbon selected from nitrogen, sulfur and oxygen.

The term “effective amount” refers to the amount of a selected compound which is effective within the context of its use or administration. In the case of therapeutic methods according to the present invention, the precise amount required will vary depending upon the particular compound selected, the age and weight of the subject, route of administration, and so forth, but may be easily determined by routine experimentation.

The term “substituted” shall mean substituted at a carbon (or nitrogen) position with, in context, hydroxyl, carboxyl, halogen, an alkyl group (preferably, C₁-C₆), alkoxy group (preferably, C₁-C₆ alkyl or aryl), ester (preferably, C₁-C₆ alkyl or aryl) including alkylene ester (such that attachment is on the alkylene group, rather than at the ester function which is preferably substituted with a C₁-C₆ alkyl or aryl group) and cyano. Preferably, the term “substituted” shall mean within its context of use alkyl, alkoxy, halogen, hydroxyl, carboxylic acid and cyano. The term unsubstituted shall mean substituted with one or more H atoms.

The term “therapeutically effective amount” or “effective amount” within context, means an amount of a compound or combination of compounds that treats a disease; ameliorates, attenuates, or eliminates one or more symptoms of a particular disease; or prevents or delays the onset of one of more symptoms of a disease. Within the context of treating cancer, especially breast cancer, the term effective amount is used to inhibit the growth and/spread of the cancer, to shrink the tumor or cancer or eliminate the cancer within the context of the cancer in the patient. Remission of the cancer is the desired outcome of this aspect of the present invention.

A “pharmaceutically acceptable salt” of the present compound generally refers to pharmaceutically acceptable salts form of a compound which can form a salt, because of the existence of for example, amine groups, carboxylic acid groups or other groups which can be ionized in a sample acid-base reaction. A pharmaceutically acceptable salt of an amine compound, such as those contemplated in the current invention, include, for example, ammonium salts having as counterion an inorganic anion such as chloride, bromide, iodide, sulfate, sulfite, nitrate, nitrite, phosphate, and the like, or an organic anion such as acetate, malonate, pyruvate, propionate, fumarate, cinnamate, tosylate, and the like. Certain compounds according to the present invention which have carboxylic acid groups may also form pharmaceutically acceptable salts, generally, as carboxylate salts, particular, sodium and potassium salts.

The term “neoplasia” is used throughout the specification to describe the pathological process that results in the formation and growth of a neoplasm, i.e., an abnormal tissue that grows by cellular proliferation more rapidly than normal tissue and continues to grow after the stimuli that initiated the new growth cease. Neoplasia could be a distinct mass of tissue that may be benign (benign tumor) or malignant (carcinoma). As used herein, the term neoplasia is used to describe all cancerous disease states and embraces or encompasses the pathological process associated with malignant hematogenous, ascitic, and solid tumors, and in particular breast cancer. The term “cancer” and the term “tumor” used in this application is interchangeable with the term “neoplasia”.

Although a number of cancers may be treated using compositions according to the present invention, the primary target of the present compounds is breast cancer, including drug resistant breast cancer. The treatment of cancer according to the present invention comprises administering to a patient an anti-cancer effective amount of one or more these agents, optionally in combination with an addition anti-cancer agent. The present invention is especially effective against breast cancer, especially drug resistant breast cancer. The treatment of breast cancer in a patient is the primary objective of this aspect of the present invention, however, the present compounds may be combined with other anti-cancer agents to treat cancer which has metastacized to other organs or tissues in the patient.

Aspects of the present invention include compounds which have been described in detail hereinabove or to pharmaceutical compositions which comprise an effective amount of one or more compounds according to the present invention, optionally in combination with a pharmaceutically acceptable carrier, additive or excipient.

Compositions according to the present invention may be administered by any conventional means known in the art. Pharmaceutical formulations include those suitable for oral, rectal, nasal, topical (including buccal and sub-lingual), vaginal or parenteral (including intramuscular, sub-cutaneous and intravenous) administration. Compositions according to the present invention may also be presented as a bolus, electuary or paste. Tablets and capsules for oral administration may contain conventional excipients such as binding agents, fillers, lubricants, disintegrants, or wetting agents. The tablets may be coated according to methods well known in the art. Oral liquid preparations may be in the form of, for example, aqueous or oily suspensions, solutions, emulsions, syrups or elixirs, or may be presented as a dry product for constitution with water or other suitable vehicle before use. Such liquid preparations may contain conventional additives such as suspending agents, emulsifying agents, non-aqueous vehicles (which may include edible oils), or preservatives. When desired, the above described formulations may be adapted to provide sustained release characteristics of the active ingredient(s) in the composition using standard methods well-known in the art.

In the pharmaceutical aspect according to the present invention, the compound(s) according to the present invention is formulated preferably in admixture with a pharmaceutically acceptable carrier. In general, it is preferable to administer the pharmaceutical composition orally, but certain formulations may be preferably administered parenterally and in particular, in intravenous or intramuscular dosage form, as well as via other parenteral routes, such as transdermal, buccal, subcutaneous, suppository or other route, including via inhalations intranasally. Oral dosage forms are preferably administered in tablet or capsule (preferably, hard or soft gelatin) form. Intravenous and intramuscular formulations are preferably administered in sterile saline. Of course, one of ordinary skill in the art may modify the formulations within the teachings of the specification to provide numerous formulations for a particular route of administration without rendering the compositions of the present invention unstable or compromising their therapeutic activity.

Compositions suitable for parenteral injection may comprise physiologically acceptable sterile aqueous or nonaqueous solutions, dispersions, suspensions, or emulsions, or may comprise sterile powders for reconstitution into sterile injectable solutions or dispersions. Examples of suitable aqueous and nonaqueous carriers, diluents, solvents, or vehicles include water, ethanol, polyols (propylene glycol, polyethylene glycol, glycerol, and the like), suitable mixtures thereof, triglycerides, including vegetable oils such as olive oil, or injectable organic esters such as ethyl oleate. Proper fluidity can be maintained, for example, by the use of a coating such as lecithin, by the maintenance of the required particle size in the case of dispersions, and/or by the use of surfactants.

These compositions may also contain adjuvants such as preserving, wetting, emulsifying, and/or dispersing agents. Prevention of microorganism contamination of the compositions can be accomplished by the addition of various antibacterial and antifungal agents, for example, parabens, chlorobutanol, phenol, sorbic acid, and the like. It may also be desirable to include isotonic agents, for example, sugars, sodium chloride, and the like. Prolonged absorption of injectable pharmaceutical compositions can be brought about by the use of agents capable of delaying absorption, for example, aluminum monostearate and/or gelatin.

Solid dosage forms for oral administration include capsules, tablets, powders, and granules. In such solid dosage forms, the active compound is admixed with at least one inert customary excipient (or carrier) such as sodium citrate or dicalcium phosphate or (a) fillers or extenders, as for example, starches, lactose, sucrose, mannitol, or silicic acid; (b) binders, as for example, carboxymethylcellulose, alginates, gelatin, polyvinylpyrrolidone, sucrose, or acacia; (c) humectants, as for example, glycerol; (d) disintegrating agents, as for example, agar-agar, calcium carbonate, potato or tapioca starch, alginic acid, certain complex silicates, or sodium carbonate; (e) solution retarders, as for example, paraffin; (f) absorption accelerators, as for example, quaternary ammonium compounds; (g) wetting agents, as for example, cetyl alcohol or glycerol monostearate; (h) adsorbents, as for example, kaolin or bentonite; and/or (i) lubricants, as for example, talc, calcium stearate, magnesium stearate, solid polyethylene glycols, sodium lauryl sulfate, or mixtures thereof. In the case of capsules and tablets, the dosage forms may also comprise buffering agents.

Solid compositions of a similar type may also be used as fillers in soft or hard filled gelatin capsules using such excipients as lactose or milk sugar, as well as high molecular weight polyethylene glycols, and the like.

Solid dosage forms such as tablets, dragees, capsules, and granules can be prepared with coatings or shells, such as enteric coatings and others well known in the art. They may also contain opacifying agents, and can also be of such composition that they release the active compound or compounds in a delayed manner. Examples of embedding compositions that can be used are polymeric substances and waxes. The active compounds can also be in micro-encapsulated form, if appropriate, with one or more of the above-mentioned excipients.

Liquid dosage forms for oral administration include pharmaceutically acceptable emulsions, solutions, suspensions, syrups, and elixirs. In addition to the active compounds, the liquid dosage form may contain inert diluents commonly used in the art, such as water or other solvents, solubilizing agents and emulsifiers, as for example, ethyl alcohol, isopropyl alcohol, ethyl carbonate, ethyl acetate, benzyl alcohol, benzyl benzoate, propylene glycol, 1,3-butylene glycol, dimethylformamide, oils, in particular, cottonseed oil, groundnut oil, corn germ oil, olive oil, castor oil, sesame seed oil, glycerol, tetrahydrofurfuryl alcohol, polyethylene glycols, fatty acid esters of sorbitan, or mixtures of these substances, and the like.

Besides such inert diluents, the composition can also include adjuvants, such as wetting agents, emulsifying and suspending agents, sweetening, flavoring, and perfuming agents.

Suspensions, in addition to the active compound, may contain suspending agents, as for example, ethoxylated isostearyl alcohols, polyoxyethylene sorbitol or sorbitan esters, microcrystalline cellulose, aluminum metahydroxide, bentonite, agar-agar, or tragacanth, or mixtures of these substances, and the like.

Compositions for rectal or vaginal administration, where applicable, can be prepared by mixing a neurotensin receptor ligand and any additional compounds with suitable non-irritating excipients or carriers such as cocoa butter, polyethylene glycol or a suppository wax, which are solid at ordinary room temperature, but liquid at body temperature, and therefore, melt in the rectum or vaginal cavity and release the neurotensin receptor ligand.

Dosage forms for topical administration include ointments, powders, sprays and inhalants. The compound(s) are admixed under sterile conditions with a physiologically acceptable carrier, and any preservatives, buffers, and/or propellants that may be required. Opthalmic formulations, eye ointments, powders, and solutions are also contemplated as being within the scope of this invention.

Seven gossypol related compounds were screened against 17betaHSD1. Gossypol resulted in only a slight reduction in enzyme activity. Four gossypol derivatives, in which the aldehyde functional group is modified, were tested. The peri-acylated nitrites, gossylic nitrile diacetate and gossylic nitrile divalerate, represent compounds in which the aldehyde group is 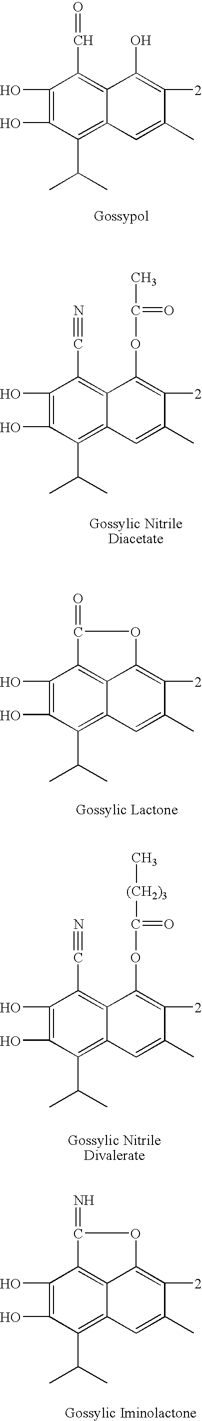
converted to a nitrile, and the peri hydroxyl of gossypol is derivatized.

Gossylic nitrile diacetate showed a 13% reduction in activity, while gossylic nitrile divalerate showed no reduction in activity at these concentrations. Gossylic iminolactone and gossylic lactone were the most promising compounds, producing 80-90% reduction in enzyme activity. The inhibition constants for gossylic lactone and gossylic iminolactone were determined to be 2.2 and 4.3 uM respectively.

Three 2,3-dihydroxynapthoic acids with different substituents at the 4- and 7-positions called gossypol analogs exhibited little effect against 17betaHSD1.

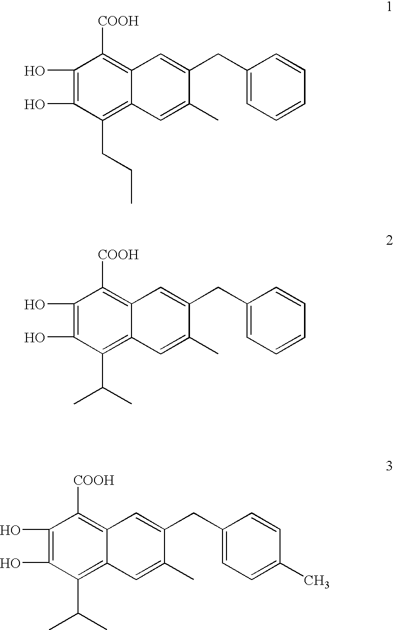

Since the gossylic lactone displayed tight binding to 17betaHSD1, flexible docking studies were performed in order to predict the binding mode of the compound. The crystal structure of 17betaHSD1 in complex with estradiol and NADP+ was used for the study. Gossylic lactone docked within the cofactor site and exhibited hydrogen bonding with several important residues as shown in figure.

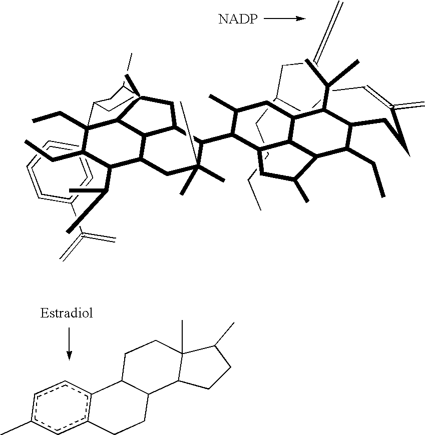

The modeling suggests that modifications of the isopropyl position with substituents can possibly take advantage of the binding region of estradiol and will lead to additional binding affinity. Substitution at this position would generate pan-active site inhibitors for 17betaHSD1. Inhibitors having the dimeric structure would be large and might present problems synthetically and may not be able to access the active site. There is also a consideration of the atropisomerism (isomers that result from hindered rotation about the binaphthyl-bond) displayed in gossylic lactone that is lacking in hemigossylic lactone. We eliminate the possibility of stereoisomers and purification of reaction products when synthesizing hemigossypol structures. To that end a hemigossylic lactone containing a substituent other than an isopropyl group was docked. The substituent contained four carbons tethered to a substrate mimetic of the C and D rings of estradiol. This resulted in a compound that docks in a pan-active orientation within the active site as shown in figure.

The docked energy of the compound is nearly doubled with respect to that of gossylic lactone. Therefore, the compound provides a promising lead compound from which synthetically accessible compounds may be derived that utilize the substrate-binding site for increased binding affinity and selectivity. 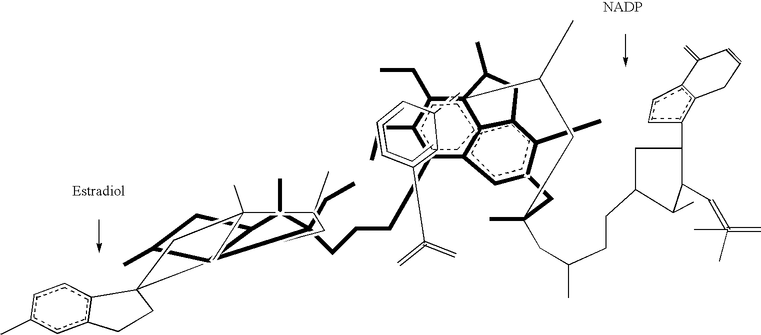
General Chemistry:

The chemistry for synthesizing compounds according to the present invention follows the general synthesis below in Scheme I. Note that the synthetic chemistry described hereinbelow, when taken with the synthetic methods which are presented in one or more of U.S. Pat. No. 4,806,568 (Gossypol Derivatives); U.S. Pat. No. 5,026,726 (Gossylic Iminolactones and Gossylic Lactones); U.S. Pat. No. 5,780,675 (Deoxygossylic Compounds); U.S. Pat. No. 5,936,12-(Deoxygossilic compounds); and U.S. Pat. No. 6,124,498 (Hydroxynapthoic acids and derivatives), which patents are incorporated by reference herein, present a facile means to compounds according to the present invention. One of ordinary skill will be able to readily modify the synthetic steps presented herein with those of the above-referenced patents in order to produce the compounds according to the present invention.

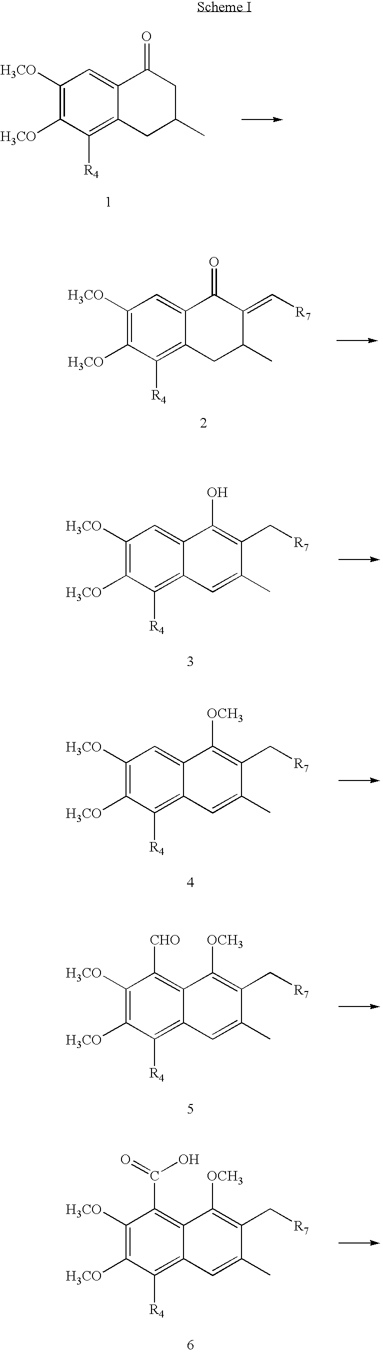

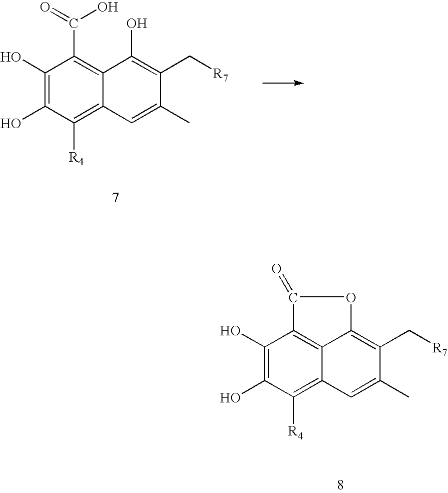

Tetralones with structure 1 have been synthesized with various alkyl groups at R4.^{1,2} Methylenation³ or aldol condensation provides unsaturated tetralones of type 2, with hydrogen, alkyl or aryl groups at R7. Heating with palladium on carbon causes compounds with structure 2 to isomerize to the corresponding naphthols (3).⁴ The naphthols will be treated with potassium carbonate and dimethyl sulfate to form trimethoxynaphthalenes (4). Compounds of structure 4 will be formylated with t-butyl lithium and N-methylformanilide according to a published procedure to provide naphthaldehydes with structure 5.⁵ The aldehyde groups of 5 will be oxidized to to carboxylic acids (6).⁶ The methyls will be removed from the phenolic hydroxyls with boron tribromide to form trihydroxynaphthoic acids (7).⁷ Compounds of structure 7 will be lactonizes with acid catalyst to form compounds of structure 8.¹ Alternative syntheses are available by analogy in U.S. Pat. No. 4,806,568 (Gossypol Derivatives); U.S. Pat. No. 5,026,726 (Gossylic Iminolactones and Gossylic Lactones); U.S. Pat. No. 5,780,675 (Deoxygossylic Compounds); U.S. Pat. No. 5,936,12-(Deoxygossilic compounds); and U.S. Pat. No. 6,124,498 (Hydroxynapthoic acids and derivatives), all of which patents are incorporated by reference herein.

Alternatively, compounds of structure 8 can be made by the efficient synthetic route shown in Scheme II. In particular, compounds of type 8 have been synthesized where R7=H. 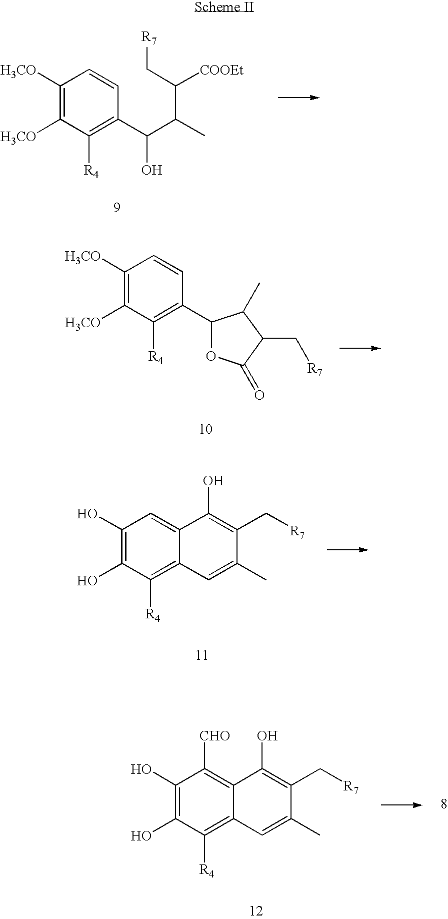
Carboxylic esters of structure 9 have been synthesized with various alkyl groups at R4 (R.^{1,2} Stirring these esters with acid catalyst causes lactonization to compounds of structure 10. The lactones of type 10 can be cyclized, aromatized and demethylated in one step with boron tribromide to form trihydroxynaphthalenes of type 11. Formylation of the trihydroxynapthalenes with titanium tetrachloride and dichloromethyl methyl ether provides naphthaldehydes of type 12. The aldehyde groups of 12 will be converted to carboxylic acids and lactonized to form compounds of type 8.¹
1) R. E. Royer, L. M. Deck, T. J. Vander Jagt, F. J. Martinez, R. G. Mills, S. A. Young and D. L. Vander Jagt (1995) Synthesis and Anti-HIV Activity of 1,1′-Dideoxygossypol and Related Compounds. J Med Chem 38, 2427-2432.
2) L. M. Deck, R. E. Royer, B. B. Chamblee, V. M. Hernandez, R. R. Malone, J. E. Torres, L. A. Hunsaker, R. C. Piper, M. T. Makler and D. L. Vander Jagt (1998) Selective Inhibitors of Human Lactate Dehydrogenases and Lactate Dehydrogenase from the Malarial Parasite Plasmodium falciparum. J Med Chem 41, 3879-3887.
3) J. L. Gras (1980) Methylene Ketones and Aldehydes by Simple, Direct Methylene Transfer: 2-Methylene-1-Oxo-1,2,3,4-Tetrahydronaphthalene. Organic Synthesis 60, 88-91.
4) H. Sheng-Yu (1987) A New Method for Synthesis of 1,6-Dimethoxy-2-isopropylnaphthalene. Acta Chimica. Sinica 45, 506-509.
5) A. Manmade, P. Herlihy, J. Quick, R. P. Duffley, M. Burgos and A. P. Hoffer (1983) Gossypol. Synthesis and In Vitro Spermicidal Activity of Isomeric Hemigossypol Derivatives. Experientia 39, 1276-1277.
6) E. Dalcanale (1986) Selective Oxidation of Aldehydes to Carboxylic Acids with Sodium Chlorite-Hydrogen Peroxide. J Org Chem 51, 567-569.
7) F. L. Benton and T. E. Dillon (1942) The Cleavage of Ethers with Boron Bromide. I. Some Common Ethers. J Am Chem Soc 64, 1128-1131.

1. A method of treating a patient for obesity comprising administering to said patient an effective amount of a compound according to the chemical structure: 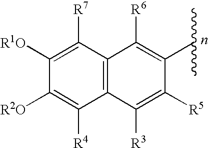
Where n is 1 or 2 such that when n is 1, n is represented as R⁹ wherein R⁹ is H, halogen, OH, OR, CO₂R group, OR₁, an optionally substituted C₁-C₈ alkyl group, an optionally substituted C₂-C₈ alkenyl or alkynyl group, an optionally substituted (CH₂)ₓcycloalkyl, (CH₂)ₓcycloalkenyl or (CH₂)ₓheterocyclic group, an optionally substituted (CH₂)ₓaryl or (CH₂)ₓheteroaryl group and when n is 2, the compound is a dimer;
R¹ and R² are independently H, an optionally substituted C₁-C₈ alkyl group, an optionally substituted C₂-C₈ alkenyl or alkynyl group, an optionally substituted (CH₂)ₓ-aryl group or (CH₂)ₓ-heteroarylgroup or a C₂-C₁₂ acyl group;
R³, R⁴ and R⁵ are independently H, halogen (F, Cl, Br, I), (CH₂)ₓOH, (CH₂)ₓCOR, (CH₂)ₓCO₂R group, (CH₂)ₓOR₁, an optionally substituted C₁-C₈ alkyl group, an optionally substituted C₂-C₈ alkenyl or alkynyl group, an optionally substituted (CH₂)ₓcycloalkyl, (CH₂)ₓcycloalkenyl or (CH₂)ₓheterocyclic group, an optionally substituted (CH₂)ₓaryl or (CH₂)ₓheteroaryl group;
R is H or an optionally substituted C₁-C₈ alkyl group;
R₁ is an optionally substituted C₁-C₈ alkyl group or C₂-C₈ acyl group or a C(O)H group;
R⁶ is H, halogen, —CN, (CH₂)ₓOH, (CH₂)ₓCOR, (CH₂)ₓCO₂R group, (CH₂)ₓOR₁, an optionally substituted C₁-C₈ alkyl group, an optionally substituted C₂-C₈ alkenyl or alkynyl group, an optionally substituted (CH₂)ₓcycloalkyl, (CH₂)ₓcycloalkenyl or (CH₂)ₓheterocyclic group, an optionally substituted (CH₂)ₓaryl or (CH₂)ₓheteroaryl group, or a 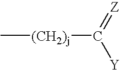
group;
Z is N—H or O;
Y is H, OH, C₁-C₈ alkyl or O(C₁-C₈)alkyl;
R⁷ is H, halogen, —CN, (CH₂)ₓOH, (CH₂)ₓCOR, (CH₂)ₓCO₂R group, (CH₂)ₓOR₁, an optionally substituted C₁-C₈ alkyl group, an optionally substituted C₂-C₈ alkenyl or alkynyl group, an optionally substituted (CH₂)ₓcycloalkyl, (CH₂)ₓcycloalkenyl or (CH₂)ₓheterocyclic group, an optionally substituted (CH₂)ₓaryl or (CH₂)ₓheteroaryl group, or a 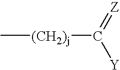
group, or R⁷ together with R⁶ forms a 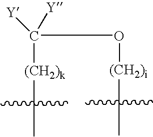
group,
where Y′ and Y″ are each H or together are O or N—H;
each x is independently 0, 1, 2, 3, 4, 5 or 6;
i is 0, 1 or 2;
j is 0, 1, 2 or 3;
k is 0, 1, 2 or 3; and
pharmaceutically acceptable salts thereof.

2. The method according to claim 1 wherein n is 1;
R¹ and R² are each independently H, or an optionally substituted C₁-C₄ alkyl group;
R³, R⁴ and R⁵ are each independently selected from H, a halogen, an optionally substituted C₁-C₄ alkyl group, an optionally substituted —(CH₂)ₓ—O(C₁-C₄)alkyl group, an optionally substituted 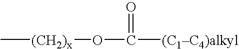
group or 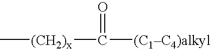
group;
R⁹ is H, a halogen, an optionally substituted C₁-C₄ alkyl group, an optionally substituted —(CH₂)ₓ—O(C₁-C₄)alkyl group, an optionally substituted 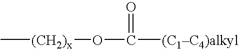
group or 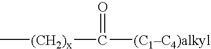
group, an optionally substituted aryl or C₁-C₆ alkylene aryl group;
R⁶ is H, OH, a halogen, an optionally substituted C₁-C₄ alkyl group, an optionally substituted —(CH₂)ₓ—O(C₁-C₄)alkyl group or together with R⁷ forms a five or six-membered lactone or lactam ring;
R⁷ is H, a halogen, an optionally substituted C₁-C₄ alkyl group, an optionally substituted —(CH₂)ₓ—O(C₁-C₄)alkyl group, an optionally substituted 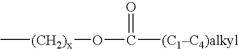
group, an optionally substituted 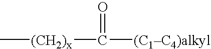
group; an optionally substituted aryl or C₁-C₆ alkylene aryl group or together with R⁶ forms a five or six-membered lactone or lactam ring.

3. The method according to claim 1 wherein R¹ and R² are each independently H, or an optionally substituted C₁-C₄ alkyl group;
R³, R⁴ and R⁵ are each independently selected from H, a halogen, an optionally substituted C₁-C₄ alkyl group, an optionally substituted —(CH₂)ₓ—O(C₁-C₄)alkyl group, an optionally substituted 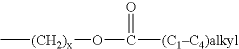
group or 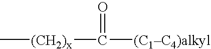
group;
R⁶ is H, OH, a halogen, an optionally substituted C₁-C₄ alkyl group, an optionally substituted —(CH₂)ₓ—O(C₁-C₄)alkyl group or together with R⁷ forms a five or six-membered lactone or lactam ring;
R⁷ is H, a halogen, an optionally substituted C₁-C₄ alkyl group, an optionally substituted —(CH₂)ₓ—O(C₁-C₄)alkyl group, an optionally substituted 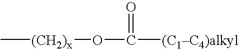
group, an optionally substituted 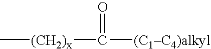
group; an optionally substituted aryl or C₁-C₆ alkylene aryl group or together with R⁶ forms a five or six-membered lactone or lactam ring; and
n is 2.

4. The method according to claim 1 wherein R¹ and R² are independently H or a C₁-C₃ alkyl group.

5. The method according to claim 2 wherein R¹ and R² are independently H or a C₁-C₃ alkyl group.

6. The method according to claim 1 wherein n is 2.

7. The method according to claim 1 wherein each x is independently 0 or 1; i is 0 or 1; j is 0 or 1; and k is 0 or 1.

8. A pharmaceutical composition comprising an effective amount of a compound according to the chemical structure: 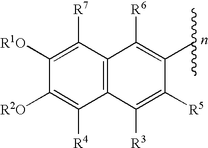
Where n is 1 or 2 such that when n is 1, n is represented as R⁹ wherein R⁹ is H, halogen, OH, OR, CO₂R group, OR₁, an optionally substituted C₁-C₈ alkyl group, an optionally substituted C₂-C₈ alkenyl or alkynyl group, an optionally substituted (CH₂)ₓcycloalkyl, (CH₂)ₓcycloalkenyl or (CH₂)ₓheterocyclic group, an optionally substituted (CH₂)ₓaryl or (CH₂)ₓheteroaryl group and when n is 2, the compound is a dimer;
R¹ and R² are independently H, an optionally substituted C₁-C₈ alkyl group, an optionally substituted C₂-C₈ alkenyl or alkynyl group, an optionally substituted (CH₂)ₓ-aryl group or (CH₂)ₓ-heteroarylgroup or a C₂-C₁₂ acyl group;
R³, R⁴ and R⁵ are independently H, halogen (F, Cl, Br, I), (CH₂)ₓOH, (CH₂)ₓCOR, (CH₂)ₓCO₂R group, (CH₂)ₓOR₁, an optionally substituted C₁-C₈ alkyl group, an optionally substituted C₂-C₈ alkenyl or alkynyl group, an optionally substituted (CH₂)ₓcycloalkyl, (CH₂)ₓcycloalkenyl or (CH₂)ₓheterocyclic group, an optionally substituted (CH₂)ₓaryl or (CH₂)ₓheteroaryl group;
R is H or an optionally substituted C₁-C₈ alkyl group;
R₁ is an optionally substituted C₁-C₈ alkyl group or C₂-C₈ acyl group or a C(O)H group;
R⁶ is H, halogen, —CN, (CH₂)ₓOH, (CH₂)ₓCOR, (CH₂)ₓCO₂R group, (CH₂)ₓOR₁, an optionally substituted C₁-C₈ alkyl group, an optionally substituted C₂-C₈ alkenyl or alkynyl group, an optionally substituted (CH₂)ₓcycloalkyl, (CH₂)ₓcycloalkenyl or (CH₂)ₓheterocyclic group, an optionally substituted (CH₂)ₓaryl or (CH₂)ₓheteroaryl group, or a 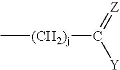
group;
Z is N—H or O;
Y is H, OH, C₁-C₈ alkyl or O(C₁-C₈)alkyl;
R⁷ is H, halogen, —CN, (CH₂)ₓOH, (CH₂)ₓCOR, (CH₂)ₓCO₂R group, (CH₂)ₓOR₁, an optionally substituted C₁-C₈ alkyl group, an optionally substituted C₂-C₈ alkenyl or alkynyl group, an optionally substituted (CH₂)ₓcycloalkyl, (CH₂)ₓcycloalkenyl or (CH₂)ₓheterocyclic group, an optionally substituted (CH₂)ₓaryl or (CH₂)ₓheteroaryl group, or a 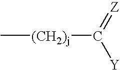
group, or R⁷ together with R⁶ forms a 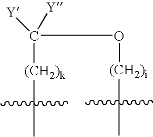
group,
where Y′ and Y″ are each H or together are O or N—H;
each x is independently 0, 1, 2, 3, 4, 5 or 6;
i is 0, 1 or 2;
j is 0, 1, 2 or 3;
k is 0, 1, 2 or 3; and
pharmaceutically acceptable salts thereof; and
b) an effective amount of a second agent useful in the treatment of obesity, diabetes (especially type II diabetes), atherosclerosis, insulin resistance, impaired glucose tolerance, hypercholesterolemia or hypertrigylceridemia, in combination with a pharmaceutically acceptable carrier, additive or excipient.

9. The composition according to claim 8 wherein said second agent is a statin compound, clofibrate, gemfibrozil or niacin.

10. A pharmaceutical composition comprising an effective amount of a compound according to the chemical structure: 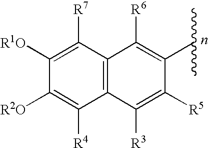
Where n is 1 or 2 such that when n is 1, n is represented as R⁹ wherein R⁹ is H, halogen, OH, OR, CO₂R group, OR₁, an optionally substituted C₁-C₈ alkyl group, an optionally substituted C₂-C₈ alkenyl or alkynyl group, an optionally substituted (CH₂)ₓcycloalkyl, (CH₂)ₓcycloalkenyl or (CH₂)ₓheterocyclic group, an optionally substituted (CH₂)ₓaryl or (CH₂)ₓheteroaryl group and when n is 2, the compound is a dimer;
R¹ and R² are independently H, an optionally substituted C₁-C₈ alkyl group, an optionally substituted C₂-C₈ alkenyl or alkynyl group, an optionally substituted (CH₂)ₓ-aryl group or (CH₂)ₓ-heteroarylgroup or a C₂-C₁₂ acyl group;
R³, R⁴ and R⁵ are independently H, halogen (F, Cl, Br, I), (CH₂)ₓOH, (CH₂)ₓCOR, (CH₂)ₓCO₂R group, (CH₂)ₓOR₁, an optionally substituted C₁-C₈ alkyl group, an optionally substituted C₂-C₈ alkenyl or alkynyl group, an optionally substituted (CH₂)ₓcycloalkyl, (CH₂)ₓcycloalkenyl or (CH₂)ₓheterocyclic group, an optionally substituted (CH₂)ₓaryl or (CH₂)ₓheteroaryl group;
R is H or an optionally substituted C₁-C₈ alkyl group;
R₁ is an optionally substituted C₁-C₈ alkyl group or C₂-C₈ acyl group or a C(O)H group;
R⁶ is H, halogen, —CN, (CH₂)ₓOH, (CH₂)ₓCOR, (CH₂)ₓCO₂R group, (CH₂)ₓOR₁, an optionally substituted C₁-C₈ alkyl group, an optionally substituted C₂-C₈ alkenyl or alkynyl group, an optionally substituted (CH₂)ₓcycloalkyl, (CH₂)ₓcycloalkenyl or (CH₂)ₓheterocyclic group, an optionally substituted (CH₂)ₓaryl or (CH₂)ₓheteroaryl group, or a 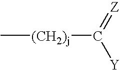
group;
Z is N—H or O;
Y is H, OH, C₁-C₈ alkyl or O(C₁-C₈)alkyl;
R⁷ is H, halogen, —CN, (CH₂)ₓOH, (CH₂)ₓCOR, (CH₂)ₓCO₂R group, (CH₂)ₓOR₁, an optionally substituted C₁-C₈ alkyl group, an optionally substituted C₂-C₈ alkenyl or alkynyl group, an optionally substituted (CH₂)ₓcycloalkyl, (CH₂)ₓcycloalkenyl or (CH₂)ₓheterocyclic group, an optionally substituted (CH₂)ₓaryl or (CH₂)ₓheteroaryl group, or a 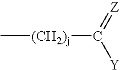
group, or R⁷ together with R⁶ forms a 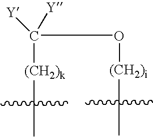
group,
where Y′ and Y″ are each H or together are O or N—H;
each x is independently 0, 1, 2, 3, 4, 5 or 6;
i is 0, 1 or 2;
j is 0, 1, 2 or 3;
k is 0, 1, 2 or 3; and
pharmaceutically acceptable salts thereof; and
b) an effective amount of a second anticancer agent, in combination with a pharmaceutically acceptable carrier, additive or excipient.

11. The composition according to claim 10 wherein said anticancer agent is selected from the group consisting of antimetabolites, etoposide, doxorubicin, taxol, vincristine, cyclophosphamide, mitomycin C, adriamycin, topotecan, campothecin, irinotecan, gemcitabine, cis-platin and mixtures thereof.

12. The composition according to claim 10 wherein said second anti-cancer agent is doxorubicin, cyclophosphamide, methotrexate and fluorouracil.

13. A method of reducing the likelihood of obesity in a patient at risk for developing obesity comprising administering an effective amount of at least one compound according to claim 1 to said patient.

14. A method of reducing the likelihood of obesity in a patient at risk for developing obesity comprising administering an effective amount of at least one compound according to claim 2 to said patient.

15. A method of reducing the likelihood of obesity in a patient at risk for developing obesity comprising administering an effective amount of a compound according to claim 3 to said patient.

16. A method of treating at least one disease state or condition in a patient in of treatment selected from the group consisting of diabetes, atherosclerosis, insulin resistance, impaired glucose tolerance, hypercholesterolemia or hypertrigylceridemia comprising administering to said patient an effective amount of a compound according to claim 1 to said patient.

17. A method of treating type II diabetes mellitus in a patient in need thereof comprising administering an effective amount of a compound according to claim 1 to said patient.

18. A method of treating breast cancer comprising administering to a breast cancer patient an effective amount of a compound or composition according to claim 1 to said patient.

19. A method of treating breast cancer comprising administering to a breast cancer patient an effective amount of a compound or composition according to claim 2 to said patient.

20. A method of treating breast cancer comprising administering to a breast cancer patient an effective amount of a compound or composition according to claim 3 to said patient.

21. A method of treating breast cancer comprising administering to a breast cancer patient an effective amount of a compound according to claim 1 in combination with an additional anticancer agent.

22. The method according to claim 21 wherein said anticancer agent is selected from the group consisting of antimetabolites, etoposide, doxorubicin, taxol, vincristine, cyclophosphamide, mitomycin C, adriamycin, topotecan, campothecin, irinotecan, gemcitabine, cis-platin and mixtures thereof.

23. The method according to claim 21 wherein said anticancer agent is selected from the group consisting of doxorubicin, cyclophosphamide, methotrexate, fluorouracil and mixtures thereof.

24. A method of inhibiting or modulating 11-β-hydroxysteroid dehydrogenase type I enzyme (11βHSD1) in a patient comprising administering to said patient an effective amount of a compound according to claim 1.

25. A method of inhibiting or modulating 17-β-hydroxysteroid dehydrogenase type I enzyme (17βHSD1) in a patient comprising administering to said patient an effective amount of a compound according to claim 1.
